(19) United States
(12) Patent Application Publication
(10) Pub. No.: US 2019/0175875 A1
(43) Pub. Date: Jun. 13, 2019

(54) Cardiovascular Catheter

(71) Applicant: Siemens Medical Solutions USA, Inc., Malvern, PA (US)

(72) Inventors: Gholamreza Mirzalou, San Jose, CA (US); Mathew Rahimi, Santa Clara, CA (US); Shahab S. Negahban, San Mateo, CA (US)

(21) Appl. No.: 15/944,839

(22) Filed: Apr. 4, 2018

Related U.S. Application Data
(60) Provisional application No. 62/596,641, filed on Dec. 8, 2017.

(51) Int. Cl. A61M 25/01; A61B 8/12; A61M 25/00; B29C 65/02; B29C 65/62; B29C 65/00

(52) U.S. Cl. CPC A61M 25/0147; A61B 8/12; A61M 25/0009; B29C 65/02; B29K 2701/12; B29C 66/52211; B29C 66/73921; A61M 2025/015; B29C 65/62

(57) ABSTRACT

A steerable catheter including a flexible elongate tube having control line lumens disposed between the inner wall surface and the outer wall surface and extending along a length of the elongate tube. The catheter further includes an anchor disposed at a distal end of the elongate tube. The catheter further includes continuous control lines, extending through the control line lumens. A portion of each of the continuous control lines is secured at the anchor so that retraction toward a proximal end of the elongate tube of ends of the continuous control lines causes movement of the distal end of the elongate tube in two directions in two orthogonal planes of movement.

### CROSS-REFERENCE TO RELATED APPLICATIONS

This application claims priority under 35 U.S.C. § 119(e) from U.S. Provisional Patent Application No. 62/596,641, filed on Dec. 8, 2017, which is hereby incorporated by reference herein in its entirety.

### FIELD OF THE INVENTION

The disclosed embodiments are directed to a cardiovascular catheter and, in particular, a cardiovascular catheter having improved anchoring of control lines.

### BACKGROUND

Ultrasound devices are an essential tool for evaluation and treatment of various diseases, especially in the field of interventional cardiology. The use of intracardiac echocardiography (ICE), with its power to visualize cardiac structures and blood flow from the inside, provides very useful support in many procedures. ICE uses a catheter-based steerable ultrasound probe that is introduced into the right heart chambers to display cardiac structures from the inside. These devices include features such as maneuverability, with the possibility of anterior/posterior and left/right deflection of the catheter.

In conventional approaches, a steerable catheter may have lumens within its walls and an anchor at the distal end thereof having corresponding lumens. Each of the lumens may carry a control line secured by a retention knot or crimping a ball at the end face of the anchor to create the resistance needed for articulation of the catheter without allowing the ends of the control lines to slip or pull out of the anchor. In such a case, the catheter wall must adequately support the anchoring knot, i.e., must be thick enough so that the knots at the ends of the control lines do not extend beyond the inner or outer wall surfaces of the catheter in a transverse direction. The outer diameter is dictated by use considerations and, in general, cannot be increased. Therefore, the use of retention knots results in a catheter having a smaller inside diameter, i.e., having a smaller central lumen. This is undesirable because the space in the central lumen is highly valuable space, as it sets the limit for available space necessary for other components/elements which may be inserted through the catheter at one time.

### SUMMARY

In one aspect, the disclosed embodiments are directed to a steerable catheter. The catheter includes a flexible elongate tube having an inner wall surface defining a central lumen and an outer wall surface defining an outer diameter of the tube, and a first pair and a second pair of control line lumens disposed between the inner wall surface and the outer wall surface and extending along a length of the elongate tube. The catheter further includes an anchor disposed at a distal end of the elongate tube, the anchor being cylindrical in shape and having an inner wall surface defining a central lumen and an outer wall surface defining an outer diameter. The catheter further includes a first and a second continuous control line, extending through the first and second pair of control line lumens, respectively. A portion of each of the first and the second continuous control lines is secured at the anchor so that retraction toward a proximal end of the elongate tube of ends of the first continuous control line causes movement of the distal end of the elongate tube in two directions in a first plane of movement, and retraction toward the proximal end of the elongate tube of ends of the second continuous control line causes movement of the distal end of the elongate tube in two directions in a second plane of movement orthogonal to the first plane of movement.

In another aspect, the disclosed embodiments are directed to a method of manufacturing a steerable catheter. The method includes providing a flexible elongate tube having an inner wall surface defining a central lumen and an outer wall surface defining an outer diameter of the tube, and further having a first pair and a second pair of control line lumens disposed between the inner wall surface and the outer wall surface and extending along a length of the elongate tube. The method further includes providing an anchor at a distal end of the elongate tube, the anchor being cylindrical in shape and having an inner wall surface defining a central lumen of the anchor and an outer wall surface defining an outer diameter of the anchor. The method further includes threading a first and a second continuous control line through the first and second pair of control line lumens, respectively. The method further includes securing a portion of each of the first and the second continuous control lines at the anchor so that retraction toward a proximal end of the elongate tube of ends of the first continuous control line causes movement of the distal end of the elongate tube in two directions in a first plane of movement, and retraction toward the proximal end of the elongate tube of ends of the second continuous control line causes movement of the distal end of the elongate tube in two directions in a second plane of movement orthogonal to the first plane of movement.

The disclosed embodiments may include one or more of the following features.

Both ends of each of the first and the second continuous control lines may extend from the first and the second pair of control line lumens at a proximal end of the elongate tube, and an approximate midpoint of each of the first and the second continuous control lines may be secured in place at the anchor. The anchor may include a first pair and a second pair of anchor control line lumens disposed between the inner wall surface of the anchor and the outer wall surface of the anchor and extending along a length of the anchor, the first pair and the second pair of anchor control line lumens being aligned with the control line lumens of the elongate tube. A first pair and a second pair of anchor threading lumens may be disposed between the inner wall surface of the anchor and the outer wall surface of the anchor and extending along a length of the anchor. The first pair of anchor threading lumens may be positioned between the first pair of anchor control line lumens in a radial direction of the anchor, and the second pair of anchor threading lumens may be positioned between the second pair of anchor control line lumens in the radial direction of the anchor.

The first and the second continuous control lines may be disposed in: the first and the second pair of control line lumens, respectively; the first and the second pair of anchor control line lumens, respectively; and the first and the second pair of anchor threading lumens, respectively. The first and the second continuous control lines may extend from the first and the second pair of control lines lumens of the anchor, may extend across a distal face of the anchor, and may extend into the first and the second pair of anchor threading lumens, respectively. The first continuous control line extends from one anchor threading lumen of the first pair of anchor threading lumens, extends across a proximal face of the anchor, and extends into another anchor threading lumen of the first pair of anchor threading lumens, the second continuous control line extends from one anchor threading lumen of the second pair of anchor threading lumens, extends across a proximal face of the anchor, and extends into another anchor threading lumen of the second pair of anchor threading lumens. A portion of the first continuous control line extending across the proximal face of the anchor may include a midpoint of the first continuous control line, and a portion of the second continuous control line extending across the proximal face of the anchor may include a midpoint of the second continuous control line. The anchor may be formed of a polymer having a higher hardness than the material of the elongate tube of the catheter. The first and the second continuous control lines may be formed of manufactured crystalline flexible fiber.

The catheter may include a control housing, wherein the elongate tube is mounted in the control housing, the control housing including a first actuator connected to both ends of the first continuous control line and a second actuator connected to both ends of the second continuous control line. The catheter may further include an anchor cap attached on at least a distal portion of the anchor, thereby securing the first and the second continuous control lines at the anchor. The anchor cap may be attached by thermoplastic melting of the anchor cap and the anchor. The first pair of control line lumens may be separated by a radial angle of about 90 degrees, and the second pair of control line lumens may be separated by a radial angle of about 90 degrees. The central lumen of the anchor may have a diameter approximately equal to a diameter of the central lumen of the elongate tube, and the outer diameter of the anchor may be approximately equal to the outer diameter of the elongate tube.

### BRIEF DESCRIPTION OF THE DRAWINGS

FIG. 1 depicts an ultrasound cardiovascular catheter assembly mounted in a control housing;

FIG. 2 is a partial cutaway view of the catheter assembly, including control lines, an anchor, and an anchor cap at the distal end thereof;

FIG. 3 is a perspective view of the distal end of the anchor threaded with the control lines;

FIG. 4 is a perspective view of the proximal end of the anchor threaded with the control lines;

FIG. 5 is a partial cutaway view of the catheter assembly, including control lines, an anchor, a cladding section, and an anchor cap at the distal end thereof;

FIG. 6 is cross-sectional view of the anchor showing control line lumens and anchor lumens disposed between the inner and outer walls of the catheter; and

FIG. 7 depicts a process for manufacturing the catheter assembly.

### DETAILED DESCRIPTION

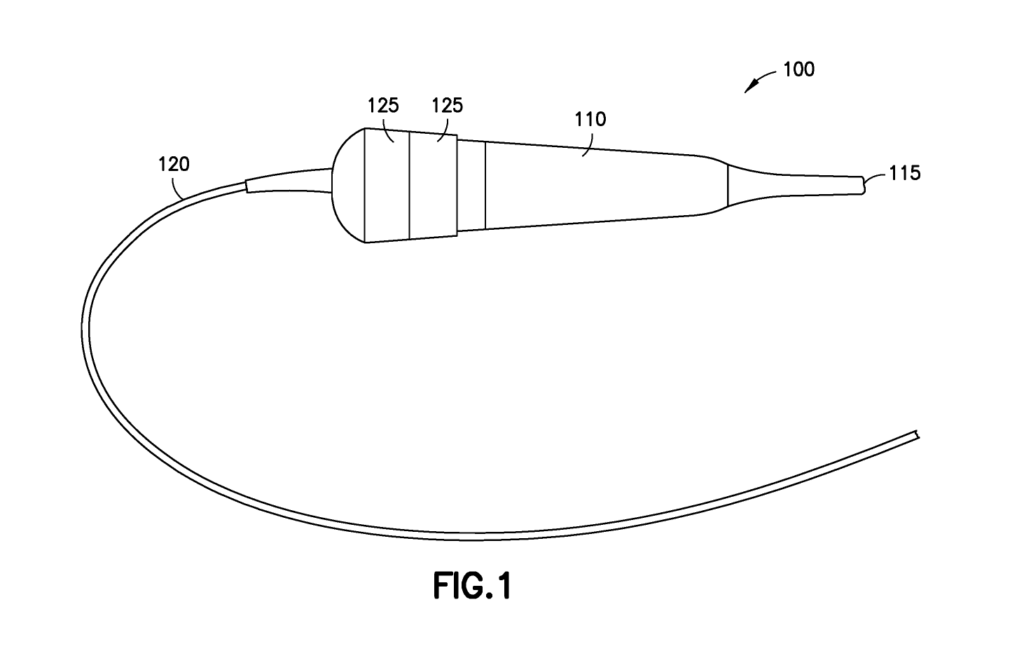

FIG. 1 depicts an ultrasound cardiovascular catheter assembly 100, which includes a catheter tube 120 mounted in a control housing 110. The control housing 110 is connected at the proximal end 115 to equipment, such as, for example, ultrasound imaging equipment (not shown). The catheter tube 120 extends from the distal end of the control housing 110. The control housing 110 includes one or more actuators 125 for applying and releasing tension to control lines (not shown) within the walls of the catheter tube 120. In disclosed embodiments, the actuators 125 may be in the form of a pair of rotating rings disposed at the distal end of the control housing 110. The rings may be configured so that rotation thereof applies tension to ends of a number of control lines. In disclosed embodiments, each actuator 125 applies tension and/or retracts the two opposite ends of a single continuous control line. For example, a first actuator 125 of the pair of actuators may be connected to the opposite ends of a first continuous control line which runs from a first point of attachment with the actuator 125, to the distal end of the catheter tube 120, and back to a second point of attachment with the actuator 125. The second actuator 125 may be similarly connected to the opposite ends of a second continuous control line. Rotating a control ring actuator 125 in one direction applies tension and/or retracts one end of its associated control line. Rotating the control ring actuator 125 in the opposite direction applies tension and/or retracts the opposite end of the associated control line.

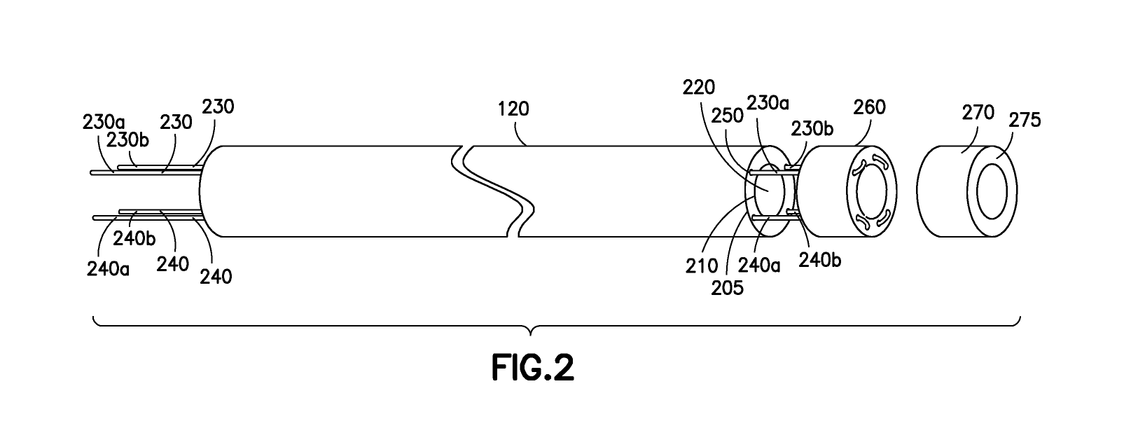

FIG. 2 is a partial cutaway view of the catheter tube 120, which has a hollow, tubular structure having an outer wall surface 205, an inner wall surface 210 and a central lumen 220 disposed within the inner wall surface 210. The catheter tube 120 may have various outer diameters depending upon the specific applications in which it is to be used. The inner diameter may be set to particular values to establish a desired wall thickness, given a determined outer diameter. In various embodiments, the catheter tube 120 may have an outer diameter of about: 8 French (inner diameter of, e.g., 52 mils), 9 Fr (inner diameter of, e.g., 73 mils), 10 Fr (inner diameter of, e.g., 55 mil), 12.5 Fr (inner diameter of, e.g., 93 mils). In disclosed embodiments, the catheter tube 120 is formed of a flexible material, such as thermoplastic material, e.g., polyether block amide (PBAX), nylon, silicone, polytetrafluoroethylene (PTFE), polyether ether ketone (PEEK), etc. The catheter tube 120 is thin and flexible enough to be inserted into the right heart chambers (e.g., the inferior vena cava) of a patient during a procedure to provide sonographic images of cardiac structures from the inside.

Control lines 230a, 230b, 240a, 240b are disposed in control line lumens 250 (for clarity, only one of the four control line lumens is labeled) running within the wall between the inner 210 and outer wall surfaces 205 of the catheter tube 120. As discussed in further detail below, the control lines 230a, 230b, 240a, 240b may be formed of a non-metallic material, such as, for example, flexible fiber. Alternatively, the control lines may be formed of a metal, such as, for example, multiple-strand stainless steel wire. In disclosed embodiments, the control lines 230a, 230b, 240a, 240b may be formed of a fiber with sufficient tensile strength to articulate the distal end of the catheter tube 120 without breaking, such as, for example, a manufactured multifilament yarn spun from liquid crystal polymer. An anchor 260 disposed at the distal end of the catheter tube 120 acts as an attachment point for the mid-points of the control lines 230a, 230b, 240a, 240b, as discussed in further detail below. An anchor cap 270 may be provided which acts as a cover for the anchor 260 after the control lines 230a, 230b, 240a, 240b have been attached to the anchor 260 (the anchor cap 270 is shown unattached to the anchor 260 in FIG. 2 for clarity).

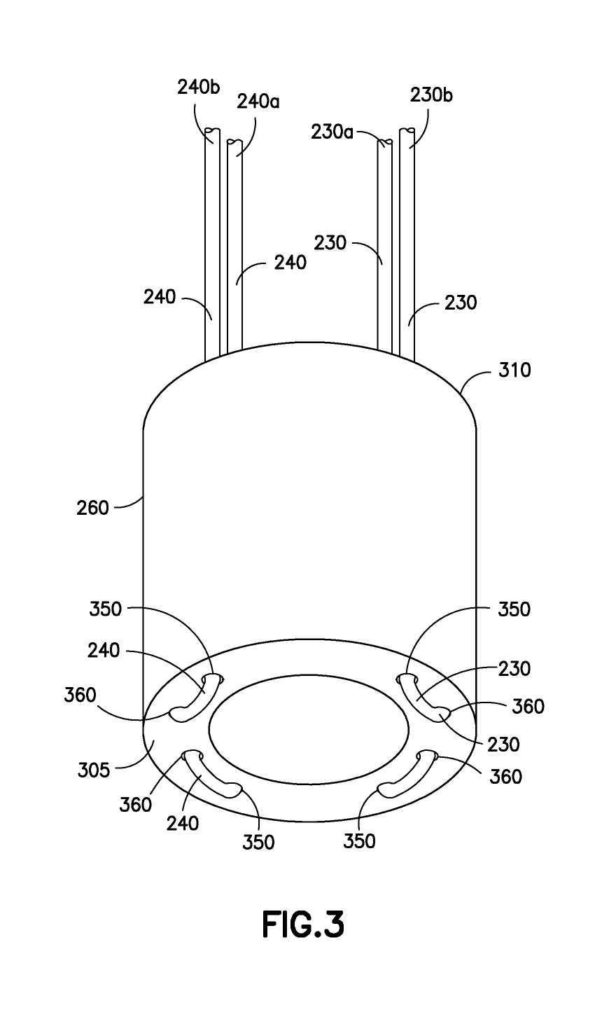

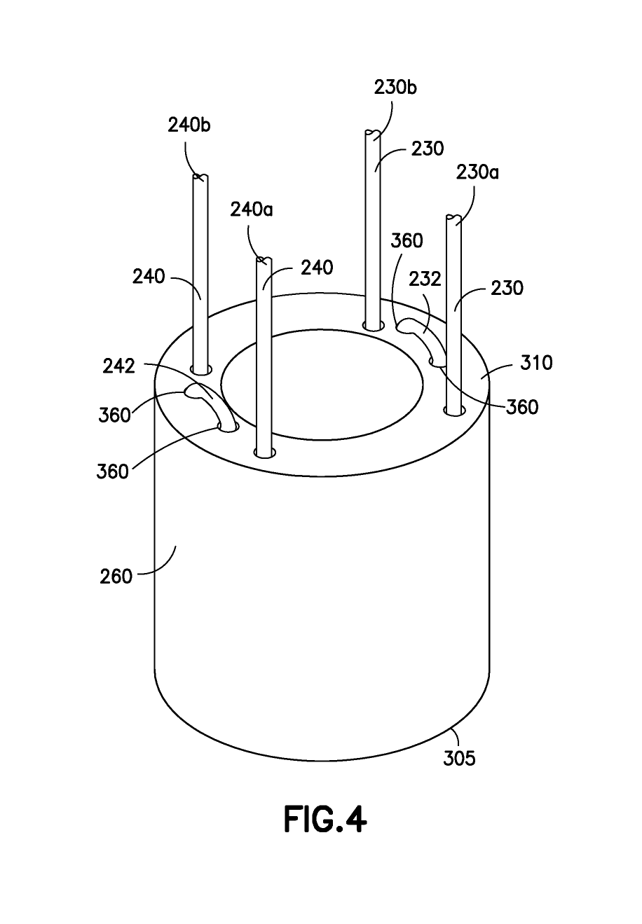

FIGS. 3 and 4 are perspective views of the distal 305 and proximal ends 310, respectively, of the anchor 260. In disclosed embodiments, there may be two continuous control lines 230, 240 which each extend from the control housing 110 to the anchor 260 and back to the control housing 110. Each of the two continuous control lines 230, 240 may be considered to have two “halves” (or “ends”) 230a, 230b, 240a, 240b which run along the length of the catheter tube 120 (the control lines may not be divided exactly in half). Consequently, there are four control line lumens 250 (see FIG. 2) which along the length of the catheter—two for each of the two continuous control lines. These four control line lumens 250 in the catheter tube 120 have corresponding control line lumens 350 in the anchor 260.

The anchor 260 may be formed (e.g., in an injection molding or continuous extrusion process) of a thermoplastic material which is harder that the material of the catheter tube 120, such as, for example, a higher hardness polymer. The control lines 230a, 230b, 240a, 240b are attached to the anchor 260 so that tension applied to the control lines 230a, 230b, 240a, 240b cause the anchor 260 to retract toward the proximal end of the catheter tube 120, thereby causing the catheter tube 120 itself to bend in a specific direction, i.e., allowing the catheter tube 120 to be “steered.”

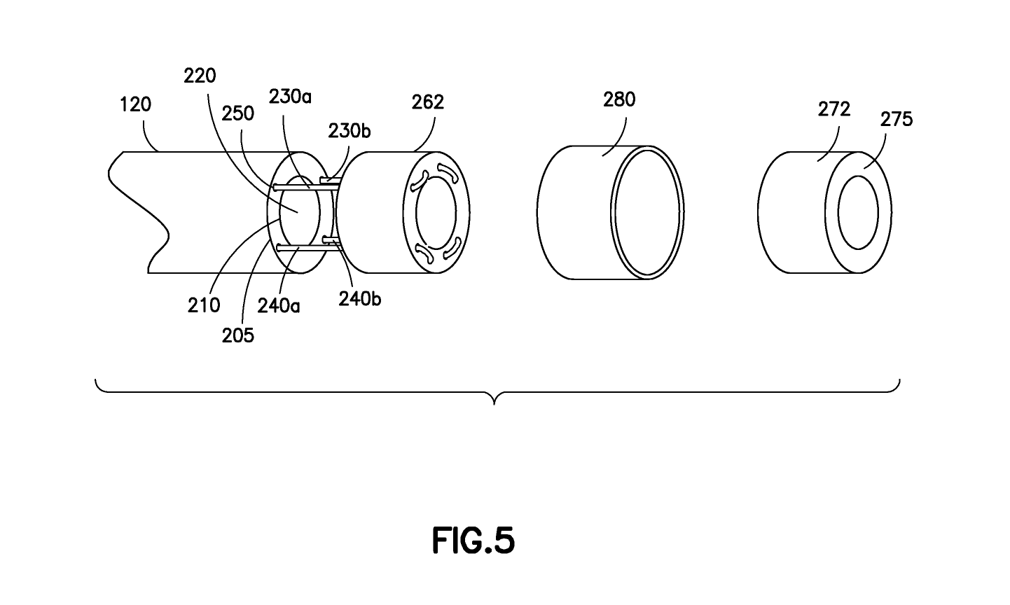

As shown in FIG. 5, in disclosed embodiments, a material may be used for the anchor 262 which does not result in melting of the anchor 262 during the manufacturing process. For example, a high temperature engineering material, such as a high-temperature resin or high-temperature thermoplastic may be used to form the anchor 262. Other materials, such as metal, may also be used. The melting range for the catheter tube 120 and anchor cap 270 thermoplastic materials is typically about 180-230° C. Therefore, a material may be selected for the anchor 262 which has a melting point which is significantly higher than this range. For example, high temperature thermoset engineering materials, e.g., polyetherimide, polyether ether ketone, polyphenylsulfone, etc., have melting temperatures higher than about 600° C. In such a case, the anchor 262 remains solid in the thermoplastic reflow (i.e., melting) process which is used to attach the anchor cap 272 and secure the control lines 230a, 230b, 240a, 240b in place. In contrast to the material used in the embodiments discussed above, which is a pliable thermoplastic material, an anchor formed of high-temperature engineering material would maintain its shape during the manufacturing process and during use. Therefore, the geometry of the anchor and anchor lumens, and the resulting sharp bends in the control lines, would remain stable while the device is in use, which would help to hold the lines within the anchor.

FIG. 5 is a partial cutaway view of the catheter tube 120. The anchor 262 is disposed at the distal end of the catheter tube 120 and acts as an attachment point for the mid-points of the control lines 230a, 230b, 240a, 240b, as in the embodiments discussed above. The anchor cap 272 acts as a cover for the anchor 262 after the control lines 230a, 230b, 240a, 240b have been attached to the anchor 262 (the anchor cap 272 is shown unattached to the anchor 262 in FIG. 2 for clarity). In disclosed embodiments, the anchor 262 is formed of high-temperature engineering material and is covered with a section of cladding 280 on the outside to prevent contact between the material and the internal tissues of the patient. The cladding section 280 may be formed, e.g., of a layer of polyether block amide (PBAX) of about 2-10 mils thickness. The outer diameter of the cladding section 280 is the same as the outer diameter of the catheter tube 120 to avoid discontinuities in the outer surface of the catheter assembly 100. Accordingly, in this embodiment, the outer diameter of the anchor is reduced by the thickness of the cladding section 280. In disclosed embodiments, the cladding section 280 may have an axial length which is the same length as the anchor 262.

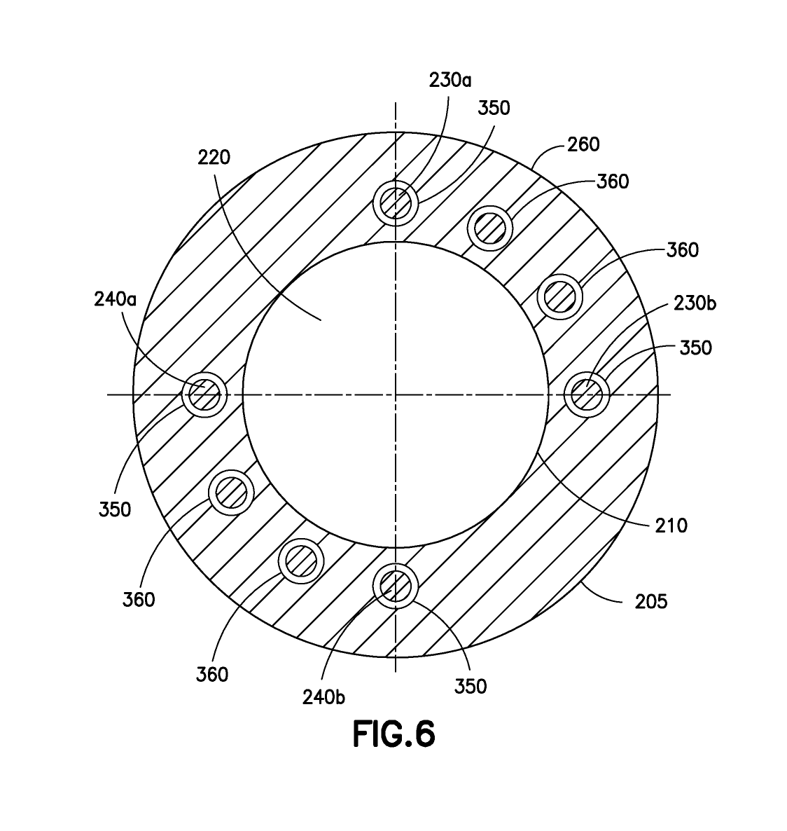

FIG. 6 is cross-sectional view of the anchor 260 showing, in accordance with disclosed embodiments, four anchor control line lumens 350 which are separated by 90 degrees and two pairs of anchor threading lumens 360 disposed between pairs of the anchor control line lumens 350. The anchor control line lumens 350 correspond in position to the control line lumens 250 in the catheter tube 120 (see FIG. 2). At the distal end of the catheter tube 120, the two ends 230a, 230b, 240a, 240b of each control line 230, 240 enter the proximal end 310 of the anchor 260 and extend completely through a pair of anchor control line lumens 350 (see FIG. 4). At the distal end 305 of the anchor 260, the ends of the control lines 230a, 230b, 240a, 240b extend from the pairs of anchor control line lumens 350, extend along the distal face 305 of the anchor 260, and extend into a pair of anchor threading lumens 360 therebetween (see FIG. 3). The control lines 230, 240 exit at the proximal face 310 of the anchor 260 such that the approximate midpoint 232, 242 of each control line 230, 240 extends between the anchor threading lumens 360 along the proximal face 310 of the anchor 260 (see FIG. 4). This configuration results in each continuous control line experiencing six 90-degree bends as it is threaded back and forth through the anchor, as well as increased surface contact between the control line and the anchor, which enhances slippage prevention.

In disclosed embodiments, the control line lumens 250 in the catheter tube 120, and the corresponding lumens 350 in the anchor 260, are positioned 90 degrees apart around the circumference of the catheter tube 120, i.e., these four lumens are “on axis.” In disclosed embodiments, the adjacent pair of anchor threading lumens 360 may be positioned so as to have a separation distance which is less than that of the corresponding anchor control line lumens 350.

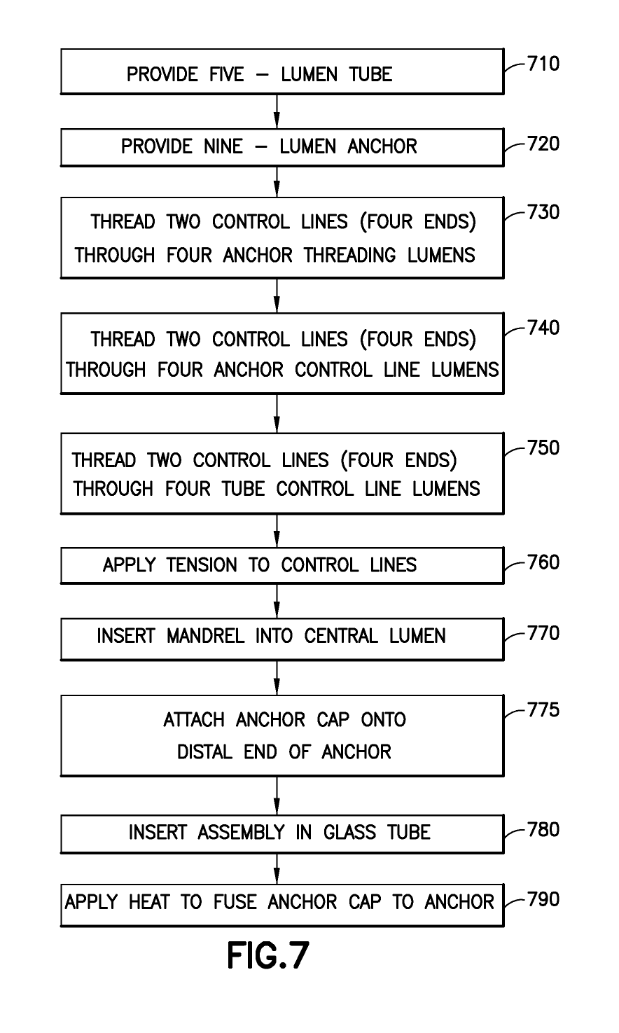

FIG. 7 depicts an embodiment of a process for manufacturing the catheter assembly 100. A thermoplastic catheter tube 120 having five lumens (including the central lumen thereof), as described above, is provided, e.g., from an extrusion process (710). A nine-lumen anchor 260 is provided, e.g., produced by an extrusion process and cut to a determined length (720) or molded. The anchor control line lumens 350 and anchor threading lumens 360 are formed within the walls of the anchor 260, resulting in the anchor 260 having nine lumens (including the central lumen of the anchor). The threading of the control lines 230, 240 is performed beginning with the anchor threading lumens 360 (730), followed by the anchor control line lumens 350 (740). The control lines 230, 240 are then threaded through the control line lumens 250 of the catheter tube 120 (750). The control lines 230, 240 are pulled until the anchor 260 abuts the distal end of the catheter tube 120 (760). Weights (e.g., 10-30 g) may be applied to the proximal ends of the control lines 230, 240 to keep them taut during the manufacturing process. The weights help to keep the control lines 230, 240 in the same relative location during attachment of the anchor cap 270, e.g., in a thermoplastic melting process. A metallic mandrel is inserted into the central lumen to maintain the shape of the central lumen during the melting process (770). A single-lumen anchor cap 270 is inserted distally against the anchor 260, thereby covering at least a distal portion of the anchor 260 (775). The catheter assembly 100 is inserted into a glass tube to maintain the shape of the outer wall of the catheter tube 120, the anchor 260, and the anchor cap 270 (780). Heat is applied at a determined temperature (based on the specific materials being used), e.g., using a hot air, infrared, conductive heating, or laser device, to melt and fuse (i.e., “reflow”) the thermoplastic material of the anchor cap 270 and the anchor 260 (790).

In alternative embodiments, one end of each of the control lines 230, 240 is threaded sequentially through the control line lumens 250 in the catheter tube 120, through the anchor control line lumens 350 and anchor threading lumens 360, and back though the control line lumens 250 of the catheter tube 120 to achieve the threading arrangement described above. The anchor cap 270 is attached as a cover for the anchor 260 and fixed in place by an attachment process, such as, for example, bonding with adhesive, fixing by tension fit between the anchor cap 270 and the anchor 260, or by a thermoplastic melting process in which the anchor cap 270 is fused with the anchor 260.

The anchor cap 270 serves as a connection element for a sensor tip (not shown), e.g., an ultrasonic transducer. In disclosed embodiments, the anchor cap 270 may be an integral part of the sensor tip. Alternatively, an anchor cap 270 having a flat distal face 275, as shown herein, may be bonded to a sensor tip. Other types of mechanical connections between the anchor cap 270 and a sensor tip are also possible As noted above, the attachment of the anchor cap 270, e.g., by thermoplastic melting, prevents the control lines 230, 240 from shifting within the lumens of the anchor 350, 360 so that tension can be independently applied to the two ends 230a, 230b, 240a, 240b of a single continuous control line 230, 240. Therefore, in disclosed embodiments, the control lines 230, 240 are thermally encapsulated within the lumens 350, 360 of the anchor 270 to prevent slippage. Thus, the two halves 230a, 230b, 240a, 240b of each control line 230, 240 may be considered to be, in effect, a separate control line. Therefore, the independent application of tension to the ends 230a, 230b, 240a, 240b of each single continuous control line 230, 240 can provide movement of the distal end of the catheter tube 120 in two directions in a single plane of motion. Because there are two such control lines, this configuration provides for the catheter to be steerable in four directions in two orthogonal planes.

Embodiments described herein are solely for the purpose of illustration. Those in the art will recognize other embodiments may be practiced with modifications and alterations to that described above.

What is claimed is:

1. A steerable catheter assembly, comprising:
a flexible elongate tube having:
an inner wall surface defining a central lumen and an outer wall surface defining an outer diameter of the tube, and
a first pair and a second pair of control line lumens disposed between the inner wall surface and the outer wall surface and extending along a length of the elongate tube;
an anchor disposed at a distal end of the elongate tube, the anchor being cylindrical in shape and having an inner wall surface defining a central lumen of the anchor and an outer wall surface defining an outer diameter of the anchor; and
a first and a second continuous control line, extending through the first and second pair of control line lumens, respectively,
wherein a portion of each of the first and the second continuous control lines is secured at the anchor so that retraction toward a proximal end of the elongate tube of ends of the first continuous control line causes movement of the distal end of the elongate tube in two directions in a first plane of movement, and retraction toward the proximal end of the elongate tube of ends of the second continuous control line causes movement of the distal end of the elongate tube in two directions in a second plane of movement orthogonal to the first plane of movement.

2. The catheter assembly of claim 1, wherein both ends of each of the first and the second continuous control lines extend from the first and the second pair of control line lumens at a proximal end of the elongate tube, and an approximate midpoint of each of the first and the second continuous control lines is secured in place at the anchor.

3. The catheter assembly of claim 1, wherein the anchor further comprises:
a first pair and a second pair of anchor control line lumens disposed between the inner wall surface of the anchor and the outer wall surface of the anchor and extending along a length of the anchor, the first pair and the second pair of anchor control line lumens being aligned with the control line lumens of the elongate tube, and
a first pair and a second pair of anchor threading lumens disposed between the inner wall surface of the anchor and the outer wall surface of the anchor and extending along a length of the anchor, the first pair of anchor threading lumens being positioned between the first pair of anchor control line lumens in a radial direction of the anchor, the second pair of anchor threading lumens being positioned between the second pair of anchor control line lumens in the radial direction of the anchor.

4. The catheter assembly of claim 3, wherein the first and the second continuous control lines are disposed in:
the first and the second pair of control line lumens, respectively,
the first and the second pair of anchor control line lumens, respectively, and
the first and the second pair of anchor threading lumens, respectively.

5. The catheter assembly of claim 3, wherein the first and the second continuous control lines extend from the first and the second pair of control lines lumens of the anchor, extend across a distal face of the anchor, and extend into the first and the second pair of anchor threading lumens, respectively.

6. The catheter assembly of claim 5, wherein the first continuous control line extends from one anchor threading lumen of the first pair of anchor threading lumens, extends across a proximal face of the anchor, and extends into another anchor threading lumen of the first pair of anchor threading lumens, the second continuous control line extends from one anchor threading lumen of the second pair of anchor threading lumens, extends across a proximal face of the anchor, and extends into another anchor threading lumen of the second pair of anchor threading lumens.

7. The catheter assembly of claim 6, wherein a portion of the first continuous control line extending across the proximal face of the anchor comprises a midpoint of the first continuous control line, and a portion of the second continuous control line extending across the proximal face of the anchor comprises a midpoint of the second continuous control line.

8. The catheter assembly of claim 1, wherein the anchor is formed of a polymer having a higher hardness than the material of the elongate tube of the catheter.

9. The catheter assembly of claim 1, wherein the first and the second continuous control lines are formed of manufactured crystalline flexible fiber.

10. The catheter assembly of claim 1, further comprising a control housing, wherein the elongate tube is mounted in the control housing, the control housing comprising a first actuator connected to both ends of the first continuous control line and a second actuator connected to both ends of the second continuous control line.

11. The catheter assembly of claim 1, further comprising an anchor cap attached on at least a distal portion of the anchor, thereby securing the first and the second continuous control lines at the anchor.

12. The catheter assembly of claim 11, wherein the anchor cap is attached by thermoplastic melting of the anchor cap and the anchor.

13. The catheter assembly of claim 1, wherein the first pair of control line lumens are separated by a radial angle of about 90 degrees, and the second pair of control line lumens are separated by a radial angle of about 90 degrees.

14. The catheter assembly of claim 1, wherein the central lumen of the anchor has a diameter approximately equal to a diameter of the central lumen of the elongate tube, and the outer diameter of the anchor is approximately equal to the outer diameter of the elongate tube.

15. A method of manufacturing a steerable catheter, the method comprising:
providing a flexible elongate tube having an inner wall surface defining a central lumen and an outer wall surface defining an outer diameter of the tube, and further having a first pair and a second pair of control line lumens disposed between the inner wall surface and the outer wall surface and extending along a length of the elongate tube;
providing an anchor at a distal end of the elongate tube, the anchor being cylindrical in shape and having an inner wall surface defining a central lumen of the anchor and an outer wall surface defining an outer diameter of the anchor;
threading a first and a second continuous control line through the first and second pair of control line lumens, respectively;
securing a portion of each of the first and the second continuous control lines at the anchor so that retraction toward a proximal end of the elongate tube of ends of the first continuous control line causes movement of the distal end of the elongate tube in two directions in a first plane of movement, and retraction toward the proximal end of the elongate tube of ends of the second continuous control line causes movement of the distal end of the elongate tube in two directions in a second plane of movement orthogonal to the first plane of movement.

16. The method of claim 15, wherein the securing of the first and the second continuous control lines is performed by attaching an anchor cap on at least a distal portion of the anchor, thereby securing the first and the second continuous control lines at the anchor.

17. The method of claim 16, wherein the attaching of the anchor cap comprises thermoplastic melting of the anchor cap and the anchor.

18. The method of claim 15, wherein the threading is performed so that both ends of each of the first and the second continuous control lines extend from the first and the second pair of control line lumens at a proximal end of the elongate tube, and an approximate midpoint of each of the first and the second continuous control lines is secured in place at the anchor.

19. The method of claim 15, wherein the anchor is formed to have:
a first pair and a second pair of anchor control line lumens disposed between the inner wall surface of the anchor and the outer wall surface of the anchor and extending along a length of the anchor, the first pair and the second pair of anchor control line lumens being aligned with the control line lumens of the elongate tube, and
a first pair and a second pair of anchor threading lumens disposed between the inner wall surface of the anchor and the outer wall surface of the anchor and extending along a length of the anchor, the first pair of anchor threading lumens being positioned between the first pair of anchor control line lumens in a radial direction of the anchor, the second pair of anchor threading lumens being positioned between the second pair of anchor control line lumens in the radial direction of the anchor.

20. The method of claim 19, wherein the threading is performed so that the first and the second continuous control lines are disposed in:
the first and the second pair of control line lumens, respectively,
the first and the second pair of anchor control line lumens, respectively, and
the first and the second pair of anchor threading lumens, respectively.

21. The method of claim 19, wherein the threading is performed so that the first and the second continuous control lines extend from the first and the second pair of control lines lumens of the anchor, extend across a distal face of the anchor, and extend into the first and the second pair of anchor threading lumens, respectively.

22. The method of claim 21, wherein the threading is performed so that the first continuous control line extends from one anchor threading lumen of the first pair of anchor threading lumens, extends across a proximal face of the anchor, and extends into another anchor threading lumen of the first pair of anchor threading lumens, the second continuous control line extends from one anchor threading lumen of the second pair of anchor threading lumens, extends across a proximal face of the anchor, and extends into another anchor threading lumen of the second pair of anchor threading lumens.
